(19) United States
(12) Patent Application Publication
(10) Pub. No.: US 2016/0158011 A1
(43) Pub. Date: Jun. 9, 2016

(54) INTRACARDIAC DEVICE TO CORRECT MITRAL REGURGITATION

(71) Applicant: Didier de Canniere, Miami Beach, FL (US)

(72) Inventor: Didier de Canniere, Miami Beach, FL (US)

(21) Appl. No.: 14/656,006

(22) Filed: Mar. 12, 2015

Related U.S. Application Data
(60) Provisional application No. 62/089,339, filed on Dec. 9, 2014.

(51) Int. Cl. A61F 2/24

(52) U.S. Cl. CPC A61F 2/2445; A61F 2230/0069

(57) ABSTRACT

A device structured to suppress mitral regurgitation by restricting prolapse of a mitral valve leaflet and including a base correspondingly dimensioned to the mitral valve and including a central portion, structured to allow blood flow there through and a peripheral portion or ring connected to the central portion in substantially surrounding relation thereto. An operative position of the base includes the central portion disposed in overlying, movement restricting relation to at least one of the valve leaflets and the ring concurrently anchored adjacent or directly to the native annulus of the mitral valve. The physical characteristics of the base facilitate its movement with and conformance to the mitral valve during diastole and systole cycles of the heart.

### CLAIM OF PRIORITY

The present application is based on and a claim of priority is made under 35 U.S.C. Section 119(e) to a provisional patent application that is currently pending in the U.S. Patent and Trademark Office, namely, that having Ser. No. 62/089,339 and a filing date of Dec. 9, 2014, and which is incorporated herein by reference.

### BACKGROUND OF THE INVENTION

1. Field of the Invention

The invention relates to a device, for use in the field of minimally invasive surgery or invasive cardiology, capable of introduction through a minimal incision, a port-access in the wall of the left atrium or via a trans-septal, catheter-based, approach to the mitral valve from a peripheral vein such as the femoral or jugular. The device is disposed that and structured to prevent a flail mitral leaflet from flipping back into the left atrium (“prolapsing”) in order to remodel the shape and movement of the mitral structures in such a way to improve the coaptation of the mitral leaflets and hence decrease or suppress the mitral regurgitation flow.

### DESCRIPTION OF THE RELATED ART

The mitral valve is located between the left atrium (LA) and the left ventricle (LV). It is due to open fully to not oppose resistance to the blood stream progressing from the LA to the LV during the diastole (i.e. the ventricular relaxation phase) and to close fully during the systole (the ventricular ejection phase) so as to prevent the blood from flowing back into the left atrium and to the pulmonary venous circulation. The role of the mitral valve is therefore to ensure antegrade progression of the blood through the left cardiac chambers. It works in synchrony with the three other heart valves that are ensuring the same function between the right atrium (RA) and the right ventricle (RV) i.e. the tricuspid valve, between the right ventricle and the pulmonary artery (PA) i.e. the pulmonic valve and downstream to the mitral valve, between the left ventricle and the aorta i.e. the aortic valve at the junction between the left ventricle and the aorta, the latter to opening during the ventricular systoles and closing during diastole. From a mechanical standpoint the mitral valve has to face high gradients of pressure during the ventricular contraction to hold up against a pressure head of about 100 mm of mercury (Hg) or more. It is recognized that the peak pressure in the LV is generally equal to or greater than 110 mmHg and the one in the atrium around 10 mmHg. This strain is absorbed mostly by the coaptation of the two mitral leaflets when closed, comprising the valve leaflets closing with each other with a contact height around 10 mm over the entire length of the mitral coaptation line. The coaptation of the leaflets depends on the adequate anatomy (integrity of the structures) and adequate functioning of 5 components, which are 1. the mitral annulus, 2. the anterior and posterior leaflets, 3. the mitral chordae, 4. the papillary muscles (PPM) and 5. the ventricular walls themselves.

Any congenital malformation or acquired lesion of one or more of these components can lead to a mitral insufficiency, also known and referred to as mitral regurgitation (MR). As commonly used, mitral insufficiency and/or mitral regurgitation is a result of the mitral valve not hermetically closing during ventricular contraction. As a result, a variable amount of blood leaks back into the LA. This situation correlates with a poor outcome for the patient, since it increases the workload to the heart, as well as it increases the volumes of the left atrial and ventricular chambers.

Furthermore, the existence of severe mitral regurgitation and ventricular dilatation generate a vicious cycle in which MR begets more MR. Indeed when the ventricle increases in size the distance between the papillary muscles increases, tethering the mitral chordae and impeding their full motion up to the plane of the annulus. The native annulus of the mitral valve may also increase. This patho-physiological continuum leads to heart failure, pulmonary hypertension, atrial fibrillation and ultimately death. The treatment of MR includes the administration of pharmacological drugs. However in most cases the regurgitation treated either by surgical repair or replacement of the valve. In some selected cases, an emerging percutaneous technology is used. However, this procedure is still under evaluation and involves the Mitraclip® or other emerging technologies that are currently under development.

Although there is a considerable trend to fix the MR as early as possible in its natural course, the indication and timing of the intervention rely also on the etiology of the condition, as well as on the functional anatomy and structural damage to the valve and the ventricle. One particular case of mitral regurgitation is referred to as structural mitral regurgitation (SMR). This includes a structural deterioration of the mitral valve and is usually the consequence of Barlow's disease or of fibro-elastic degeneration (FED). This condition is extremely prevalent and can be found, according to different studies, in about 2-4% of the adult population.

Repairing structural mitral insufficiency poses particular problems and challenges that have been approached in different ways. Such include a surgical approach through various incisions in the patients' chest using cardiopulmonary bypass (CPB) on the arrested heart. Less frequently the approach involves, percutaneously using an endovascular, catheter that requires, a trans-septal puncture. The trans-septal puncture involves drilling a hole in the inter-atrial septum in order to reach the left atrial chamber of the heart from the punctured vein. This maneuver requires sophisticated infrastructures and highly trained teams and can be applied only in carefully selected, hence limited, subcategories of patients.

Surgery is currently regarded as the golden standard of treatment to repair the mitral valve and is therefore performed in the vast majority of the cases. When the valve is repaired technical failure is not a rare event as up to 20% of the patients who undergo repair experience recurrence of severe mitral regurgitation during the first post-operative year. In a significant number of cases of SMR, generally less than 50%, the leaking valve is replaced rather than repaired. This occurs for numerous reasons including technical difficulties and insufficient physician's experience/caseload. Replacement represents a loss of chances for the patient as compared to repair with an estimated increase in the mortality risk around 15% at five years after the operation for SMR. In any case open heart surgery remains a major acute insult to patients' physiology with risks of complications arising mainly from three maneuvers: sternal division (“sternotomy”), CPB and aortic clamping/manipulations. Generally such an operation corresponds in terms of bodily inflammatory response to that of a third degree burn of 25% of the body surface area.

Therefore, an alternative solution allowing an easier, less invasive, more reproducible, and possibly safer and more durable reduction or disappearance of the mitral regurgitation is needed to overcome the problems as generally set forth above.

### SUMMARY OF THE INVENTION

This invention is directed to the use of an intra-cardiac pre-shaped device, where in one or more preferred embodiments comprise a grid tailored or more specifically corresponding in dimension and configuration to patient's mitral valve anatomy. As such, the intracardiac device of the present invention includes a base having a dimension and configuration which corresponds to that of the native annulus and leaflets of the mitral valve.

More specifically, the device of the present invention comprises a base including a peripheral portion connected in at least partially surrounding relation to a central portion. As indicated, the base and or its components may be pre-formed and structured prior to its application to correspond in both dimension and configuration to the mitral valve including the native annulus thereof. Such preoperative structuring may be in accord with a three-dimensional (3-D) print of the patient's mitral valve. As such, the patient's mitral valve, using any of a plurality of appropriate imaging techniques, may be “reconstructed” in three dimensions, in order to assure an accurate, customized dimensioning and configuring of the base. Such imaging techniques are known in the medical profession and related prior art and may include, but are not limited to, a CT scan, MRI, 3D echo imaging, etc. The preoperative dimensioning and configuring of the base of the device facilitates its securement in an appropriate operative position relative to the mitral valve being treated. As set forth above, the base comprises a peripheral portion and a central portion secured to the peripheral portion and being surrounded thereby. The central portion comprises a grid or open mesh configuration or other appropriate structure which facilitates the flow of blood through the central portion. Moreover, the grid or open mesh configuration comprises a plurality of openings which are collectively disposed, dimensioned and configured to facilitate the aforementioned normal blood flow there through, from the left atrium to the left ventricle. Such facilitated blood flow is necessary due to the operative positioning of the base in an overlying relation to the mitral valve substantially or at least partially on the interior of the left atrium. As a result, during the normal functioning of the heart, blood will flow through the grid or open mesh of the central portion, through the open orifice of the mitral valve and into the left ventricle, when the heart is in diastole.

In addition, the central portion including the grid or open mesh configuration will also be disposed in overlying, movement restricting relation to at least one of the leaflets of the mitral valve, when the base is in the aforementioned operative position. Therefore at least a part of the central portion will be disposed in engaging relation with at least one of the valve leaflets preferably, but not necessarily exclusively, at an area in overlying alignment with the regurgitating orifice. As used herein, the “regurgitating orifice” is intended to describe the opening between the leaflets of the mitral valve through which blood flows from the left ventricle back into the left atrium during diastole. As explained in greater detail hereinafter, the grid or open mesh configuration may include the aforementioned plurality of openings extending over a predetermined part of the central portion or at least a majority of the central portion. In at least one embodiment, substantially the entirety of the central portion is comprised of the plurality of openings which facilitate the aforementioned blood flow, during an open orientation of the mitral valve, into the left ventricle. As will be explained in greater detail hereinafter, the open mesh construction of the central portion will still provide sufficient resistance to at least one of the leaflets to restrict its movement back into the left atrium. Accordingly, it is emphasized, that the intended structural and operative features of the base, being correspondingly dimensioned and configured with mitral valve being treated, facilitates both blood flow through the mitral valve as well as the restriction of movement or prolapse of at least one valve leaflet. As a result, the device of the present invention, when operatively positioned relative to the mitral valve will restrict or at least decrease the propensity for mitral regurgitation.

As indicated, the base also comprises a peripheral portion which preferably includes an annular configuration and/or ring structure. The ring structure is anchored adjacent to the native annulus of the mitral valve and/or directly thereto such that the grid or open mesh of the central portion is disposed in overlying relation to the valve leaflets of the mitral valve. Further, the material from which the peripheral portion or ring of the base is formed may be accurately described as being “semi-rigid”. As used with regard to the physical characteristics of the ring, the term semi-rigid is meant to include a material having sufficient flexibility facilitate movement of the ring with the native annulus between the open and closed orientations of the mitral valve. At the same time, the semi-rigid material of the ring preferably includes sufficient rigidity to restrict or limit an abnormal or undesired dilation of the native annulus, when the ring is anchored to and/or adjacent the native annulus, while the base is in the operative position.

An additional structural feature of the intracardiac device includes the ring having a length which is equal to at least a majority and preferably substantially the entirety of the circumference of the native annulus. As such, at least a majority or substantially the entire length of the ring is anchored directly or adjacently along at least a majority or preferably the entirety of the circumference of the native annulus. Also, in order to provide more accurate fitting or attachment of the ring relative to the native annulus, the ring may not have a continuous configuration. More specifically, the ring may include free opposite ends which when in an operative position, are disposed in adjacent but spaced relation to one another.

Structural and operative features of the grid or open mesh of the central portion may include it being formed of a material having sufficient flexibility to move with the mitral valve as it is disposed between the open and closed orientations. However, the central portion should also include sufficient rigidity, strength, tenacity, etc. to restrict movement of at least one of the valve leaflets and prevent prolapse thereof into the left atrium as the mitral valve assumes a closed or orientation during systole. To this end, the central portion, including the open mesh grid may have a substantially “bowed”, at least partially “domed” or similar, outwardly projecting configuration. In more specific terms, such a preferred bowed or domed configuration of the central portion facilitates it at least partially entering the orifice of the mitral valve at least when the mitral valve is in an open orientation. However, upon a closing of the mitral valve the bowed configuration may at least minimally retract or otherwise be reoriented such that it remains in engaging and/or movement restricting relation to at least one leaflet of the mitral valve at least during systole in order to prevent the aforementioned prolapse thereof.

Introduction of the device may be accomplished by an introductory catheter passing through the left atrial wall. As at least partially indicated above, the flexibility of both the peripheral portion or ring and the central portion of the base is such as to allow it to be initially disposed in a folded, crimped or other reduced volume orientation. When so oriented within the interior, the base will be disposed within the interior of the introductory catheter to be delivered to the interior of the left atrium. In addition, an additional catheter or positioning instrument may also be disposed on the interior of the introductory catheter in associated relation with the base. Once the introductory catheter is disposed within the interior the left atrium, the positioning instrument forces the device out through an access opening of the introductory catheter. Further, the flexible and/or semi-rigid structuring of the material of the device may also include a sufficient “inherent bias”. As such the base will automatically expand into its intended configuration for anchoring and/or placement in the operative position relative to the mitral valve.

As indicated, the central portion is secured to the peripheral portion ring and is surrounded thereby. In turn, when applied in its operative position, the ring is anchored adjacent to or indirect attachment with the natural annulus of the mitral valve. Such anchoring may occur through appropriate suturing or through the utilization of a plurality of anchoring hooks or other appropriate connectors which will securely connect and maintain the peripheral portion ring in the operative position relative to the native annulus.

Additional features relating primarily, but not exclusively to the application of the base to the mitral valve may include a separate attachment or anchoring of both the peripheral portion ring and the central portion. More specifically, the central portion ring can be initially applied into the interior of the left atrium and anchored in its intended location relative to the natural annulus. Thereafter, the central portion may be entered into the left atrium and connected about its periphery to the peripheral portion ring in a secure and reliable manner. In contrast to the above, the central portion and the peripheral portion ring may be connected to one another pre-operatively and prior to introduction into the left atrium.

Therefore, different structural lesions can affect the anatomy of the mitral valve leading to mitral regurgitation. The device of the present invention, as described herein is primarily targeted at fixing “flail” leaflets i.e. leaflets that are “prolapsed” because of chordal rupture and/or extension also overcome and/or restrict mitral regurgitation from other etiologies. More, technically the device of the present invention corresponds to a type II according to Carpentier's classification. It is an extremely frequent phenomenon.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic representation of an open orientation of the mitral valve orifice.

FIG. 2 is a schematic representation of a closed orientation of the mitral valve orifice.

FIG. 3 is a perspective view in schematic form of an anterior leaflet of the mitral valve in prolapse further demonstrating mitral regurgitation.

FIG. 4 is a perspective view in schematic form of the device of the embodiment the device in an operative position overlying the leaflets of the mitral valve.

FIG. 5 is a perspective view in schematic form of normal blood flow from the left atrium through the mitral valve, where in the device of the embodiment of FIGS. 1 and 4 are in an operative position.

FIG. 6 is a perspective view in schematic form of the peripheral portion of the base of the embodiment of FIGS. 3-5 secured in an operative position to the native annulus of the mitral valve.

FIG. 7 is a perspective view in schematic form of the device of the embodiment of FIGS. 1-4 in a position for attachment in to the mitral valve using a plurality of connectors.

FIG. 8 is a perspective view of the device of FIGS. 1-7 disposed in an operative position relative to the mitral valve leaflets when in a closed orientation.

FIG. 9 is a perspective view of the device of FIGS. 1-7 disposed in an operative position relative to the mitral valve orifice, when in an open orientation.

FIG. 10 is a perspective view in schematic form of one method and/or procedure for inserting the device of the present invention into the left atrium in preparation for attachment to the mitral valve in an operative position.

FIG. 11 is a perspective view in schematic form of the embodiment of FIG. 10 wherein the device has exited an introductory catheter and is disposed in position for attachment to the mitral valve.

Like reference numerals refer to like parts throughout the several views of the drawings.

### DETAILED DESCRIPTION OF THE PREFERRED EMBODIMENT

As represented in the accompanying Figures, the present invention is directed to an intracardiac device generally indicated as 10, which is structured to restrict prolapse of a mitral valve leaflet, as at 104, and by doing so restrict or diminish mitral valve regurgitation, as schematically represented in FIG. 3. More specifically, the base 12 includes an outer peripheral portion 14 having a substantially annular configuration. As such the peripheral portion 14 may include a ring structure. As also represented the central portion 16 is connected along its outer circumference to the peripheral portion 14 so as to be substantially or at least partially surrounded thereby, as clearly represented in the Figures. As also represented, the peripheral portion ring may not be continuous. More specifically, in order to facilitate the disposition of the ring 14 in the preferred operative position, the opposite ends may be disposed in adjacent but spaced relation to one another when in the operative position, relative to the native annulus 126.

Further, the central portion 16 of the base 12 comprises a grid construction or configuration which is more specifically defined as an open mesh construction or configuration. As such, the grid or open mesh of the central portion 16 comprises a plurality of openings cooperatively disposed, dimensioned and configured to facilitate the passage of fluid, specifically including blood, there through, as will be explained in greater detail hereinafter with primary reference to FIG. 5.

While the general configuration of the peripheral ring portion 14 is represented in the Figures, the base 12 and its components may be formed and/or structured preoperatively so as to correspond in both dimension and configuration to the mitral valve 100, including the native annulus 102 thereof, to which it is applied. Such preoperative structuring may be in accord with a three-dimensional (3-D) replica of the patient's mitral valve 100. As such, required dimensions and/or configuration of the patient's mitral valve 100 may be determined using a variety of scanning techniques known in the medical profession and related arts. Moreover, the generally customized dimensioning and configuration of the base 12 of the device 10 facilitate its securement in an intended operative position relative to the mitral valve 100 being treated.

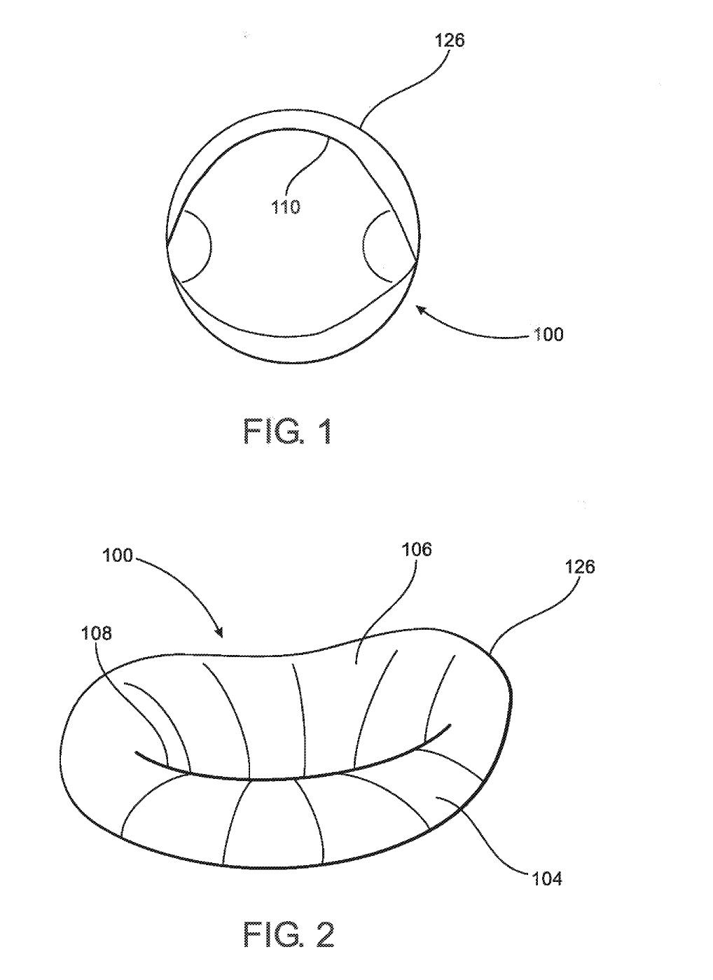

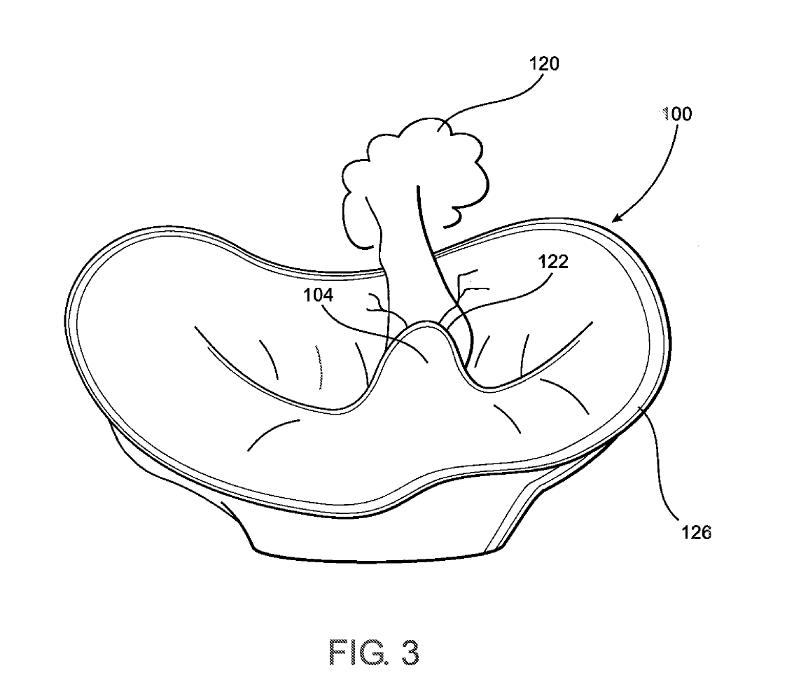

In the normal functioning of the heart, the mitral valve 100 will repetitively move between an open orientation, as represented in FIG. 1, and a closed orientation, as represented in FIG. 2. As such, when in a closed orientation the valve leaflets 104 and 106 close along a coaptation line 108. In contrast, when in an open orientation the natural orifice 110 of the mitral valve 100 is open to facilitate blood flow there through from the left atrium to the left ventricle during diastole of the heart. Accordingly, the plurality of openings of the grid or open mesh which at least partially define the central portion 16 may extend across substantially the entirety thereof or at least along a major portion thereof. However the collective disposition of the plurality of openings must be such as to assure adequate blood flow through the mitral valve 100 from the left atrium to the left ventricle. It is also noted that certain segments or parts of the open mesh of the central portion 16 may be structured to include a greater density. Such increased density may be defined by the plurality of openings in the dense segment greater in number and more closely positioned. This increased density may further facilitate the movement restricting engagement or relation of the central portion 16 relative to one or more of the valve leaflets 104 or 106.

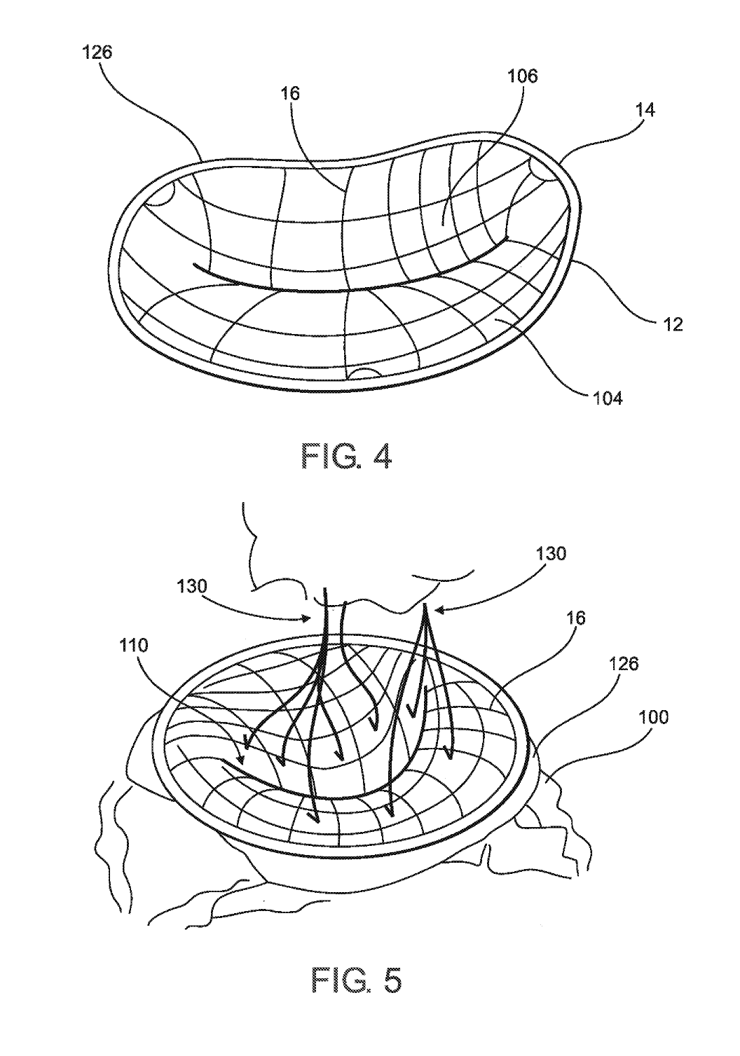

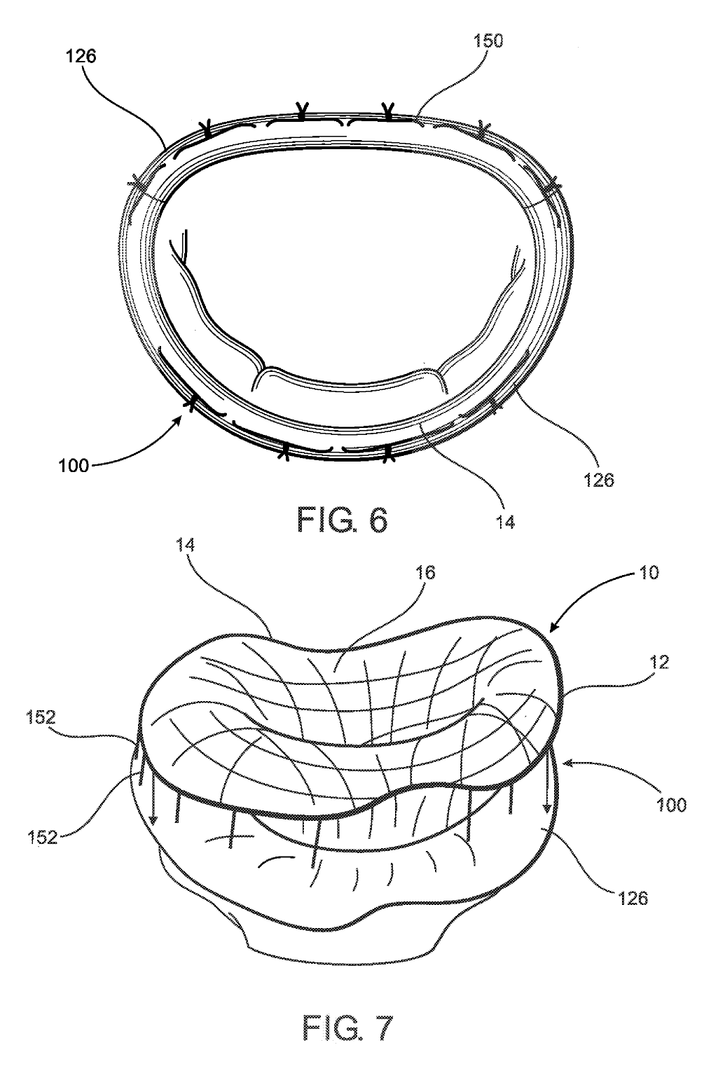

As indicated and with reference to FIGS. 3-5, the device 10, including the base 12, is disposed in an operative position so as to restrict movement of at least one of the valve leaflets 104 and 106. In restricting movement or prolapse of a corresponding leaflet, mitral regurgitation, as schematically represented FIG. 3, may be eliminated or reduced. As represented an anterior leaflet 104 of the mitral valve 100 is in a state of prolapse as it moves back into the left atrium. As a result, mitral regurgitation occurs allowing a flow of blood 120 back into the left atrium through a regurgitation orifice 122. Therefore, the preferred operative position of the base 12 is schematically represented in FIGS. 4 and 6. As such, the peripheral portion or ring 14 is secured adjacent or directly to the natural annulus 126 of the mitral valve 100. In such an operative position the central portion 16, including the grid or open mesh, is disposed in overlying relation to one or both of the mitral valve leaflets 104 and 106. Further, the base 12 will remain connected in its operative position when the mitral valve 100 is in the closed position of FIG. 4 as well as the open position of FIG. 5. The overlying disposition and movement restricting engagement of the open mesh of the central portion 16 will also remain in movement restricting engagement with the one or more valve leaflets 104 and 106 during both the closed an open orientation of the mitral valve 100. As schematically demonstrated in FIG. 5, when in the open orientation (also see FIG. 1), the plurality of openings which at least partially define the central portion 16 facilitates blood flow generally indicated as 130 from the left atrium, through the open mesh and/or grid of the central portion 16 and through the natural mitral orifice 110 of the mitral valve 100.

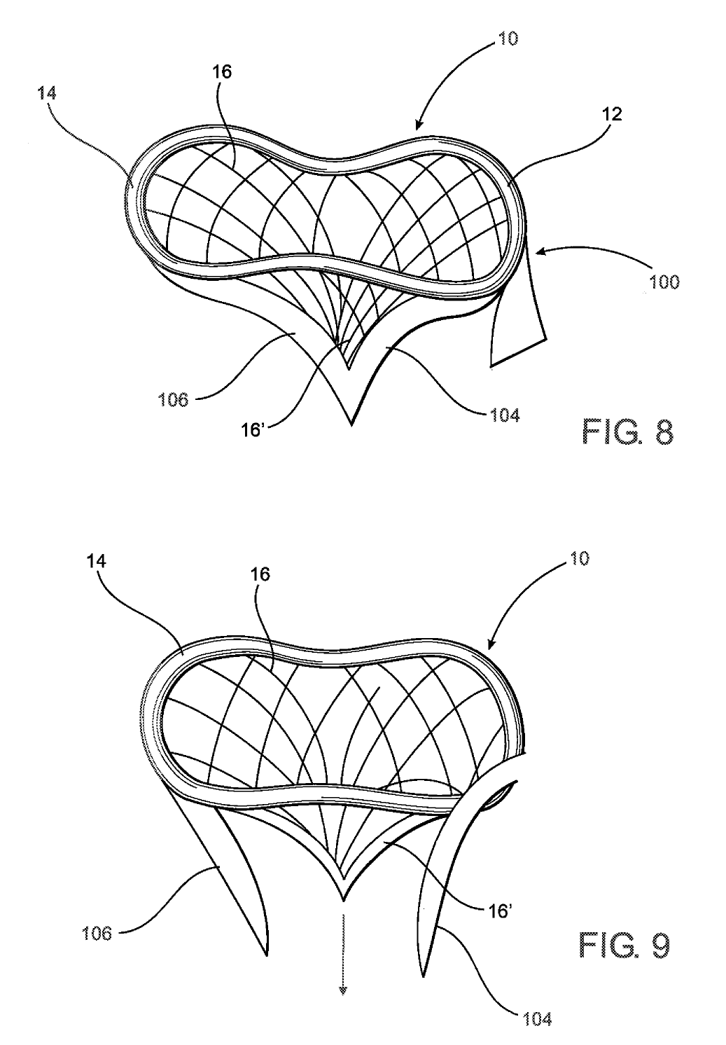

As schematically represented in FIGS. 6 and 7, attachment of the peripheral portion or ring 14 adjacent or directly to the natural annulus 126 may be accomplished by suturing as at 150. In the alternative, a plurality of hook like connectors or other appropriately structured connectors 152 may be affixed to the peripheral portion 14 in spaced relation to one another. As applied, the plurality of connectors will be secured directly to the natural annulus 126 or in a sufficiently adjacent location to dispose the peripheral portion 14 in the manner demonstrated in FIGS. 7 and 8. Therefore, when properly attached to the mitral valve 100 in the operative position, the base 12 will have both sufficient flexibility and rigidity to move with the mitral valve 100, including the natural annulus 126, between the open and closed orientations as represented in FIGS. 1 and 2.

In more specific terms, the material from which the peripheral portion or ring 14 of the base 12 is formed may be accurately described as being “semi-rigid”. As used herein, this term with specific regard to the peripheral portion ring 14 is meant to include a material having both a degree of flexibility and a degree of rigidity. Moreover, the material of the peripheral portion ring 14 is sufficiently flexible to facilitate movement of the ring 14 with the native annulus 126 between the open and closed orientations of the mitral valve 100. At the same time, the semi-rigid material of the peripheral portion ring 12 preferably includes a sufficient rigidity to restrict or limit a predetermined and/or abnormal or undesired dilation of the native annulus 126 upon movement of the natural orifice 110 into the open orientation, when the ring 14 is anchored to or adjacent the native annulus 126, as represented throughout the Figures. As set forth above, such an abnormal dilation or expansion may occur because of an existing mitral regurgitation condition.

Somewhat similarly, the structural and operative features of the grid or open mesh of this central portion 16 includes a sufficient amount of rigidity, strength and tenacity to restrict movement of at least one of the valve leaflets 104 and 106. Such restricted movement prevent prevents or reduces prolapse of the one or more leaflets 104 and 106 as the mitral valve 100 assumes a closed orientation during systole. At the same time, the grid or open mesh of the central portion 16 should have sufficient flexibility to accommodate and move with the different orientations of the mitral valve 100 including the natural orifice 110 and the native annulus 126. Therefore, with primary reference to FIGS. 7-9, the grid or open mesh of the central portion 16 may include a substantially “bowed” or “domed” configuration generally indicated as 16′. As such, the bowed configuration 16′ is disposed, dimensioned and configured to be disposed in movement restricting engagement with one or both of the leaflets 104 and 106 of the mitral valve 100 when in the closed orientation of FIG. 7. Also, the substantially bowed or domed configuration 16′, as well as the flexibility of the central portion 16, will facilitate at least a partial insertion or passage of the bowed segment 16′ through or into the natural orifice 110 of the mitral valve 100 when the mitral valve 100 is in the open orientation of FIG. 9.

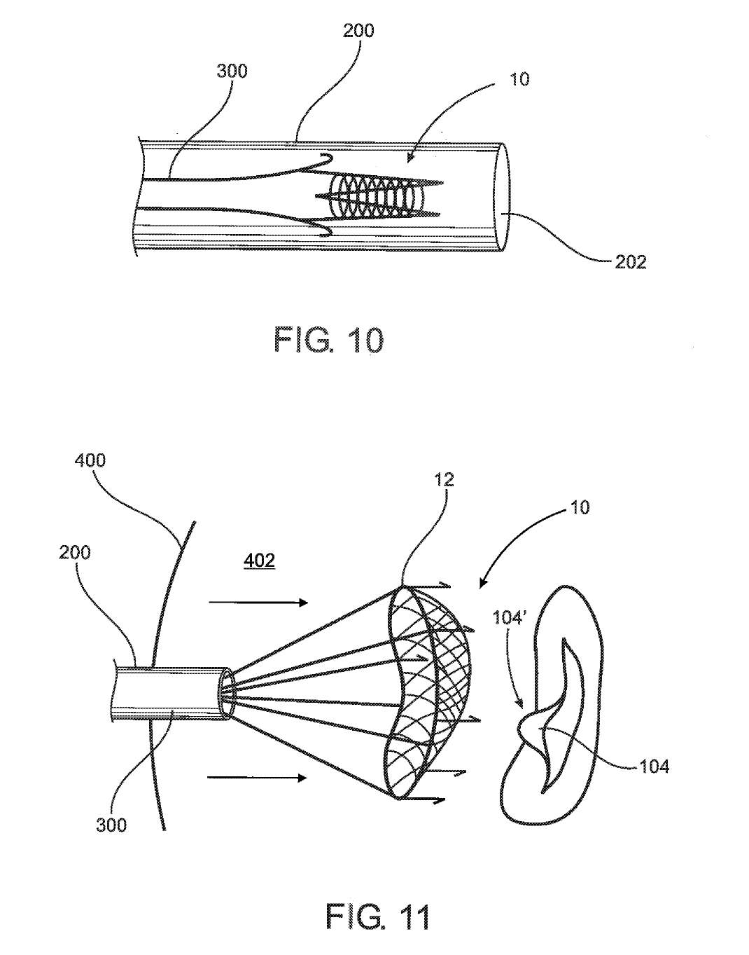

With primary reference to FIGS. 10 and 11, introduction of the device 10 may be by and introductory catheter 200 passing through a wall 400 of the left atrium 402. As generally indicated above, the flexibility and overall structuring of the device 10 allows it to be folded or otherwise manipulated into a reduced volume orientation of sufficiently reduced size to be disposed and moved within the interior of the introductory catheter 200. Also, a positioning instrument 300 may also be operatively disposed within the interior of the introductory catheter 200 in direct association with the device 10. Further manipulation of the positioning instrument 300 will cause the device 10 to pass through and out of an access opening 202 once the introductory catheter 200 passes through the atrium wall 400 into the interior of the left atrium 402. Accordingly, as represented in FIG. 12, additional manipulation of the positioning instrument 300 will result in a forced removal of the device 10 from the introductory catheter 200. As also indicated above, the flexibility and other physical characteristics of the material from which the components of the base 12 are formed may also be such as to include an inherent bias. As a result, once the device 10 exits the access opening 202 it will assume its normal expanded orientation, as represented. Once in the normal or expanded orientation the device 10 and base 12 will be disposed relative to the mitral valve 100 for attachment thereto in the aforementioned operative position. For purposes of clarity, the FIG. 12 also represents the mitral valve 100 having at least one leaflet 104 being disposed in prolapse as at 104′.

Additional features relating primarily, but not exclusively, to the insertion and attachment of the base 12 to the mitral valve 100 may include a separate attachment of the peripheral portion ring 14 and a separate or subsequent attachment of the central portion 16. In contrast, the central portion 16 and the peripheral portion ring 14 may be connected to one another preoperatively and prior to disposition of the devise 10 into the introductory catheter 200, as described above.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A device structured to restrict prolapse of a mitral valve leaflet into the left atrium of the heart, said device comprising:
a base including a peripheral portion and a central portion collectively disposed in an operative position relative to the mitral valve,
said central portion connected to and surrounded by said peripheral portion,
said central portion at least partially including a liquid permeable construction,
said base movable with the mitral valve between an open orientation and a closed orientation of the mitral valve when in said operative position, and
said operative position comprising said peripheral portion secured adjacent to a native annulus of the mitral valve and said central portion disposed in overlying, movement restricting relation to at least one mitral valve leaflet.

2. The device as recited in claim 1 wherein said operative position further comprises said peripheral portion secured adjacent the native annulus within the left atrium.

3. The device as recited in claim 1 wherein said peripheral portion is at least partially formed of a material having sufficient flexibility to move with the annulus between the open and closed orientations of the mitral valve.

4. The device as recited in claim 1 wherein said liquid permeable construction comprises a grid configuration structured and disposed to facilitate blood flow there through from the left atrium through an orifice of the mitral valve into the left ventricle during a diastole phase of the heart.

5. The base as recited in claim 4 wherein said operable position further comprises said grid configuration disposed in aligned, overlying relation to a coaptation line of the mitral valve leaflets, at least during a systole phase of the heart.

6. The device as recited in claim 4 wherein said grid configuration comprises a plurality of openings extending over a portion of said base, said plurality of openings disposed in aligned, overlying relation to the mitral orifice of the mitral valve.

7. The device as recited in claim 6 wherein a segment of said base and corresponding ones of said plurality of openings are disposed in aligned, overlying relation with a coaptation line of the mitral valve leaflets, said corresponding plurality of openings including a greater density than other portions of said grid configuration.

8. The device as recited in claim 1 wherein at least a portion of said central portion comprises a substantially bowed configuration.

9. The device as recited in claim 8 wherein said bowed configuration comprises at least a portion of said grid configuration extending from said left atrium into movement restricting engagement with at least one mitral valve leaflet, at least during a systole phase of the heart cycle.

10. The device as recited in claim 1 wherein said base comprises a bowed configuration comprising at least a portion of said grid configuration protruding from the left atrium, at least partially through the mitral orifice, at least during a diastole phase of the heart cycle.

11. The device as recited in claim 10 wherein said bowed configuration further comprising at least a portion of said grid configuration extending from the left atrium into movement restricting engagement with at least one mitral valve leaflet, at least during a systole phase of the heart cycle.

12. The device as recited in claim 1 where in at least a majority of said central portion comprises a grid configuration.

13. The device as recited in claim 12 wherein substantially an entirety of said central portion comprises said grid configuration; an outer periphery of said grid configuration connected to said peripheral portion.

14. The device as recited in claim 1 wherein said peripheral portion comprises a ring anchored adjacent to a native annulus of the mitral valve along at least a majority of a length of said ring, said ring formed of a sufficiently flexible material to move with the native annulus between open and closed orientations of the mitral valve.

15. The device as recited in claim 14 wherein said ring includes a non-continuous configuration having oppositely disposed free ends; said operative position further comprising said free ends disposed on the native annulus in adjacent, spaced relation to one another.

16. The device as recited in claim 14 wherein said ring includes a longitudinal dimension sufficient to extend along at least a majority of the length of the native annulus when anchored adjacent thereto in said operative position.

17. The device as recited in claim 16 wherein said ring is formed of a material having sufficient rigidity to restrict a predetermined, abnormal dilation of the native annulus, at least during diastole of the heart.

18. The device as recited in claim 1 wherein said peripheral portion and said central portion are cooperatively dimensioned and configured to collectively correspond to a native annulus and valve leaflets of the mitral valve, when in an open configuration and a closed configuration.

19. A device structured to restrict prolapse of a mitral valve leaflet into the left atrium of the heart, said device comprising:
a base correspondingly dimensioned to the mitral valve and including a central portion and a ring,
said ring connected to said central portion in substantially surrounding relation thereto and said base concurrently disposed in overlying relation to the mitral valve when in an operative position,
said operative position further comprising said ring anchored adjacent to a native annulus of the mitral valve and said central portion disposed in movement restricting relation to at least one mitral valve leaflet, at least during a systole phase of the heart, and
said central portion structured to facilitate blood flow there through during a diastole phase of the heart.

20. The device as recited in claim 19 wherein said ring is formed of a sufficiently flexible material to move with the native annulus between open and closed orientations of the mitral valve, when anchored adjacent thereto in said operative position.

21. The device as recited in claim 20 wherein said ring includes a longitudinal dimension sufficient to extend along at least a majority of the length of the native annulus when anchored adjacent thereto in said operative position.

22. The device as recited in claim 20 wherein said central portion comprises an open mesh configuration including a plurality of openings collectively dimensioned to facilitate blood flow through said central portion.

23. The device as recited in claim 22 wherein said operative position further comprises at least a portion of said open mesh configuration disposed in overlying relation to the coaptation line and in movement restricting relation to at least one leaflet of the mitral valve concurrent to the systole phase of the heart.

24. The device as recited in claim 23 wherein at least some of said plurality of openings are in aligned, fluid communicating relation with an orifice of the mitral valve when in an open orientation.

25. The device as recited in claim 22 wherein said open mesh configuration extends over at least a majority of said central portion.

26. The device as recited in claim 20 wherein said central portion is formed of a substantially semi-rigid material having sufficient rigidity to restrict prolapse of the at least one mitral valve leaflet during a systole phase of the heart, when said base is in said operative position.

27. The device as recited in claim 19 wherein said semi-rigid material of said central portion includes sufficient flexibility to at least partially protrude from the left atrium into a mitral orifice of the mitral valve, at least during a diastole phase of the heart cycle.

28. The device as recited in claim 19 wherein at least a part of said central portion comprises a substantially bowed configuration, said bowed configuration comprising at least a part of said central portion extending from the left atrium into movement restricting engagement with at least one mitral valve leaflet, at least during a systole phase of the heart cycle.

29. A device structured to restrict prolapse of a mitral valve leaflet into the left atrium of the heart, said device comprising:
a base correspondingly dimensioned to the mitral valve and including a central portion and a ring,
said ring at least partially defining an outer circumference of said base and disposed in surrounding relation to said central portion,
said operative position further comprising said ring anchored at least adjacent to a native annulus of the mitral valve,
said ring dimensioned and structured to restrict a predetermined, abnormal dilation of the native annulus, at least during a diastole phase of the heart, and
said central portion disposed to receive passage of blood flow there through during a diastole phase of the heart.

30. The device as recited in claim 29 wherein said ring is formed of a sufficiently flexible material to move with the native annulus between open and closed orientations of the mitral valve, when anchored adjacent thereto in said operative position.

31. The device as recited in claim 29 wherein said ring includes a longitudinal dimension sufficient to extend along at least a majority of the length of the native annulus when anchored adjacent thereto in said operative position.
